(19) United States
(12) Patent Application Publication
(10) Pub. No.: US 2005/0250138 A1
(43) Pub. Date: Nov. 10, 2005

(54) Process for the enumeration and identification of microorganisms

(75) Inventors: Barbara Young, Lexington, MA (US); Andrew Sage, Littleton, MA (US)

Correspondence Address: MILLIPORE CORPORATION, 290 CONCORD ROAD, BILLERICA, MA, 01821, US

(73) Assignee: Millipore Corporation, Billerica, MA (US)

(21) Appl. No.: 11/090,119

(22) Filed: Mar. 25, 2005

Related U.S. Application Data
(60) Provisional application No. 60/567,953, filed on May 4, 2004.

(51) Int. Cl. C12Q 1/68; C12P 19/34

(52) U.S. Cl. 435/6; 435/91.2

(57) ABSTRACT

A test for the detection, enumeration and identification of microorganisms in a fluid sample is taught. The test uses a filter through which a fluid sample is passed and onto which organisms within the fluid are deposited. It is then subjected to an amplification procedure with a reagent containing one or more detector agents. Thereafter the filter is subjected to a presence/absence test for organisms. The same sample and filter is simultaneously or subsequently subjected to a detection test to identify targeted organisms.

### CROSS REFERENCE RELATED APPLICATIONS

This application claims the benefit of U.S. Provisional Application No. 60/567,953, filed on May 4, 2004.

### BACKGROUND OF THE INVENTION

The traditional method of determining the presence or absence of microorganisms in liquids such as water, food products such as juices or pharmaceuticals has been to filter the fluid through a suitable membrane to trap any microorganisms present in the fluid on the surface of the membrane. The membrane is then placed on a growth media plate such as a Petri Dish filled with agar or other suitable media and then incubated for several days to allow colonies to develop from the captured organisms.

The plates are then removed and examined visually so that the number of colonies present can be counted. If the number is high enough, further tests may be conducted to determine exactly what organisms are present. (Often the mere presence of organisms is not in and of itself an indication that the fluid is unsafe and further work to identify the specific organisms is required.).

This process can typically span from two to fourteen days or more depending upon the organisms, their prescribed incubation time and growth rate and the additional identification tests that need to be run as well as when a sample was taken during the work day and when it will be ready on the next available work day.

Recently, new tests for the detection and enumeration of microbial contaminants have helped reduce the time required to conduct the tests to about 24 hours and even sometimes less. For example, ATP bioluminescence is a biochemical reaction that produces light energy as a product; this method has been used to detect and enumerate microbes in ⅓ the time of growth needed for the visual detection on media. Other technologies utilize fluorescent molecules, or other stains to rapidly detect microorganisms from a variety of samples on membranes. Probe hybridization technology has also been used to detect and enumerate microorganisms on membranes in shorter time frames than growth to colonies; in these cases, microbes that had been grown on a membrane for a short time are hybridized with nucleic acid probes and treated with a detection reagent to detect any micro-colonies present on the membrane surface.

U.S. 2003-0003540-A1 improves upon the ATP tests. It captures microbe on a membrane and incubates the organisms on a media for several hours to a day. An enumeration test is first conducted (generally a bioluminescent ATP detection) and then PNA probes are used in a second test on the same sample to determine the identification of the microbes found.

None of these tests however allow one to have a test that detects, enumerates and identifies the microorganisms in real time (within a few hours) with the same accuracy and specificity of the classic multiday visual test. The present invention provides a single test that detects and enumerates the microorganism in a matter of a few hours on a membrane surface, and also allows the possible identification as well in the same procedure.

### SUMMARY OF THE INVENTION

The present invention relates to a process for enumerating and identifying microorganisms. More particularly, it relates to a process for amplifying specific components of the microbes present and then enumerating them on a membrane device in a single step with minimal or no incubation time based on detection of the amplified component product. Additionally, the microbes present may be identified by this method.

The present invention relates to a process for the detection, enumeration and identification of microorganisms in a fluid using membrane filtration, targeted amplification and detection such as by fluorescent detection or calorimetric detection. A key attribute of the present process is that the amplification step allows for the rapid detection, enumeration and identification of organisms that are present. The amplification targets a select molecule in the organism such as its DNA or RNA and increases the presence of that molecule to a level that is detectable by any number of available detection technologies in order to confirm that the target molecule is present. The amplification reagent contains one or more detection agents to indicate the presence of a target molecule(s) and allow one to differentiate between selected targets on a species or genus or even phylum, kingdom level.

It is an object of the present invention to provide a process for enumerating and identifying microorganisms comprising:
a) filtering a liquid sample through a membrane suitable for the retention of microorganisms,
b) subjecting the microorganisms on the membrane to an amplification step using one or more reagents containing one or more detection agents, and
c) determining the presence/absence of one or more retained microorganisms, counting the number detected and identifying the type of microorganism present by the presence of the selected detector agents.

### BRIEF DESCRIPTION OF THE DRAWINGS

FIG. 1 shows a block diagram of a preferred embodiment of the present invention.

FIG. 2 shows a block diagram of a second preferred embodiment of the present invention.

### DETAILED DESCRIPTION OF THE SPECIFICATION

The present invention relates to a process for the rapid detection, enumeration and identification of microorganisms in fluids or soluble solids such as powders, salts, creams, tablets, etc. Such fluids include but are not limited to water, potable or otherwise, dairy such as milk, beer, wine, soft drinks, fruit juices, pharmaceuticals, parenterals, bacterial air counts, etc.

Current rapid technology allows one to detect and enumerate microorganisms in far less time than the classic methods of growth to visible colonies on agar or membrane filters. However, those organisms detected remain unidentified. This idea addresses the need for an immediate detection and enumeration system, and allows the possible identification of the detected organisms with specific probes at species or genus grouping in the amplification reactions, or at higher levels with phyla, or class specific probes.

The product visualized is a membrane-associated, targeted amplification reaction such as, but not inclusive of, the polymerase chain reaction (PCR) based method coupled to a membrane composed of hydrophilic domains bound by hydrophobic gridding such as is used in the MicroStar® system available from Millipore Corporation of Billerica, Mass. There are several components: a hydrophilic membrane covered with a hydrophobic gridding, reagents to carry out the targeted amplification consisting of nucleic acid, or peptide nucleic acid primers, enzymes, buffers and detergents, a fluorescent detection probe built into the amplification cocktail, and a mylar or plastic sleeve in which the reaction would be performed (chemoluminescent or chromogenic based detection systems may also be employed). The devices required are a temperature control system to maintain the temperature appropriate for targeted amplification reactions; for example, a temperature cycler would be needed for PCR reaction. Also, a detection device to count hydrophilic wells on the gridded membrane exhibiting a positive signal is required. The detection device could be as simple as a handheld UV light, or as complex as a MicroStar device.

The product can be used in the following manner. A sample containing microorganisms would be filtered through the gridded membrane and contaminant organisms captured. After rinsing, the amplification reagents would be sprayed onto the membrane surface, and the membrane would be placed into the Mylar® film sleeve and sealed. The sealed sleeve with the membrane and reagents would then be placed in device to control the environment of the amplification reaction. For example, for a PCR reaction, this would consist of a temperature cycler. After completion, the sample would be placed into the detection device. The device would detect those wells containing a positive signal produced by the fluorescent detection probe, or some like reagent. A positive signal would be consistent with the presence of a targeted microbial contaminant. Tests could be specific for microorganisms at the species, genus or group level inclusive of bacteria, yeast and mold, viruses, and protozoa. For example, the test could target the nucleic acid of the bacterial species Pseudomonas aeruginosa, all true pseudomonads, or broadly targeting all gram-negative bacteria, or all bacteria. This would allow one to obtain a measure of different types of organisms in the test sample, and determine the identity of any particular microbes of interest to the user.

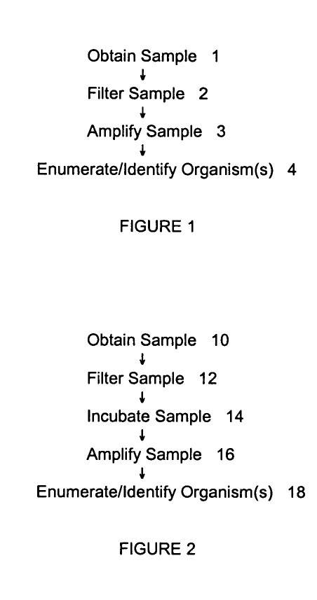

FIG. 1 shows a first preferred process according to the present invention. In this process, as the first step 1, a sample in fluid form is obtained from the product to be diagnosed. For example, in environmental sampling, such as a water supply, a sample of water from the river, well or reservoir is obtained. For a food or pharmaceutical product, a sample is taken from the stock (typically this is the finished stock although it need not be so). If in liquid form it is simply used as is or may, if necessary, due to viscosity be diluted with deionized water. If in solid form, it is dissolved or dispersed in a suitable liquid, typically water or an alcohol.

It is then filtered through a membrane filter in the second step 2 and the filter is then treated in step 3 with an amplification agent that contains one or more selected detector agents. The filter is then placed into an amplification device, such a thermal cycler for PCR amplification, for a time sufficient to amplify the sample to a level that can be detected or enumerated in step 4 either through a mechanical or electrical device or visually.

FIG. 2 shows a second process that first obtains a sample 10 and filters the sample to retain the microbes on the filter surface 12 as in the embodiment of FIG. 1. Unlike the embodiment of FIG. 1, this embodiment adds an incubation step 14 before the amplification step 16 so as to allow a micro-colony to develop from the individual retained microbes thus increasing the number of organisms available for amplification. Following the amplification step, the organisms are enumerated and/or identified in step 18.

A preferred device for holding the filter is a MILLIFLEX™ filter funnel having a 50 mm diameter and a 100 ml capacity, available from Millipore Corporation of Billerica, Mass. Other devices such as the Steritest™ or Sterifil® filtration units available from Millipore Corporation of Billerica, Mass. that have hydrophobic grids formed on and through a hydrophilic membrane added may also be used, or membrane holders such as glass or stainless steel filter holders or funnels. Such devices are well known and available from a variety of sources including Millipore Corporation of Billerica, Mass. and Fisher Scientific, Inc, of Pittsburgh, Pa.

A preferred membrane is a MicroStar™ filter, available from Millipore Corporation of Billerica, Mass. This filter is a 0.45 nominal pore size PVDF filter having a series of hydrophilic compartments separated by hydrophobic partitions that extend through the entire depth of the filter. Other filters that may be useful in this invention would also be hydrophilic and contain some type of hydrophobic partitioning.

As discussed in relation to the embodiment of FIG. 2 above, the filtered sample may be subject to an incubation step 14. Preferably, it is accomplished by placing the filter in contact with a growth media such as various agars or liquid media. The incubation is a short period of time, typically from about 1 to about 8 hours, preferably from about 1 to about 4 hours. The incubation allows the single microbes captured on the membrane to divide into a greater number of organisms for amplification making detection faster and easier, especially when the target in the organisms is difficult to amplify.

The filter is subjected to the amplification step for a short period of time, typically from 1 to 5 hours. The length of time depends upon the accuracy needed by the test, the rate of amplification for the amplification technology selected, the detection agent(s) selected, the method for detecting the agent (mechanical or human eye), the type of organism to be detected, the desired speed of the test vs. the accuracy of the test results and other such factors.

Suitable methods for amplification include nucleic acid based systems such as polymerase chain reaction (PCR) amplification in which a DNA or RNA sequence in the contaminating cell is amplified by an amplification agent. The PCR is typically conducted by placing the substrate (in this case a filter) containing the organism to be amplified, and having been treated with amplification agent during cycling to elevated temperatures for a period of time from about 30 minutes to 90 minutes in a thermal cycler. Commercially available kits for conducting a PCR amplification step are available from Applied Biosystems, Inc. or Roche Diagnostics. Thermal cyclers are available from these same companies.

Other possible amplification technologies include but are not limited to nucleic acid sequence based amplification (NASBA) available from Biomerieux, strand displacement amplification (SDA) available from Becton Dickenson, or linked linear amplification (LLA) available from Biorad. Also see EP 1407050A2.

It should be understood that the assays run herein with amplification step are on either single cells or micro-colonies of microorganisms that develop from the single cells captured on the membranes, especially if an incubation step is used, i.e. one is enumerating the numbers of single organisms in a test sample by detection of targeted molecules that have been amplified from those same single organisms rather than detecting the individual organisms directly.

After a suitable time period for the filter in the amplification step, the filter is removed and subjected to an enumeration step 4.

A preferred enumeration test is by fluorescence detection of the amplified target. The target molecule is then detected through the detector agent incorporated into the amplification reagent such as a fluorescent tag, a radioactive tag, a colorimetric tag and the like. The agent is then read visually or digitally through an imagining device such as CCD camera with image processor. One such system is sold as MicroStar™ system, available from Millipore Corporation of Billerica, Mass. By whatever means that is used, the count and location of the microbial colonies detected can be made.

During the enumeration test, the sample can also subjected to an identification test for the target organisms. They may be designed so as to specifically hybridize only to a single species or to an entire genus. The detector agents contain a tag such as an enzyme, hapten, fluorophore or radioisotope to indicate their presence in the sample. Such agents are available from a variety of sources including Sigma Genosys, and may consist of molecular beacons, dual labeled fluorescent probes, or fluorescent labeled probes to describe fluorescent probes, though chemiluminescent, calorimetric, or radiological detection systems may be used as well.

One may select one or more of such agents to detect one or more types of organisms. Some exist for individual organisms such as E. coli or Salmonella sp., L. brevis, etc, while others are more universal and simply detect the presence of the genus of the bacteria or whether the organism that has been captured is simply a bacteria, a yeast or fungi. Depending upon agent selected, one can use X-ray film, a fluorescent, or calorimetric detection or other such device to detect the presence of the tag of the agents which have been amplified with the target molecule such as the DNA or RNA of the organism and thereby identify the number and type of each target organism present.

1) A process for detecting, enumerating and identifying microorganisms comprising: (a) filtering a liquid sample through a membrane suitable for the retention of microorganisms, (b) subjecting the membrane to an amplification step wherein the amplification step contains one or more detection agents and (c) detecting the presence/absence of one or more microorganisms, the number detected and type.

2) The process of claim 1 wherein the liquid sample is from 50 to 1000 milliliters, the membrane is selected from the group consisting of PVDF membrane having hydrophilic areas separated by hydrophobic partitions, the amplification step occurs form a period of time from about 30 to about 90 minutes and the detection is by a device suitable for the chosen detection agent(s).

3) The process of claim 1 wherein the amplification is a nucleic acid-based amplification system and the detection agent is selected from the group consisting of a fluorescent-labeled nucleic acid, peptide nucleic acid probe and mixtures thereof and is read by digital imaging system.

4) The process of claim 1 wherein the amplification is a nucleic acid-based amplification system in the form of polymerase chain reaction (PCR) and the detection agent is selected from the group consisting of a fluorescent-labeled nucleic acid, peptide nucleic acid probe and mixtures thereof and is read by digital imaging system.

5) The process of claim 1 wherein the amplification is a nucleic acid-based amplification system, the reagent being selected from the group consisting of a DNA target and a RNA target and the detection agent is selected from the group consisting of a fluorescent-labeled nucleic acid, peptide nucleic acid probe and mixtures thereof and is read by digital imaging system.

6) The process of claim 1 wherein the amplification is a nucleic acid-based amplification system in the form of polymerase chain reaction (PCR), the reagent being selected from the group consisting of a DNA target and a RNA target and the detection agent is selected from the group consisting of a fluorescent-labeled nucleic acid, peptide nucleic acid probe and mixtures thereof and is read by digital imaging system.

7) The process of claim 1 wherein the detection agent is read by a process selected from the group consisting of X-ray film, visual detection of a colorimetric reaction on the membrane, visual detection of a fluorescent tag or by digital imaging.

8) The process of claim 1 wherein the amplification step is selected from the group consisting of polymerase chain reaction (PCR), nucleic acid sequence based amplification (NASBA), strand displacement amplification (SDA) and linked linear amplification (LLA) and the membranes are incubated from about 30 minutes to about 90 minutes at a temperature of from about room 25° C. to about 90° C. in a thermal cycler or other temperature controlled device.

9) The process of claim 6 wherein the membranes are incubated from 1 to about 4 hours before amplification.

10) The process of claim 1 wherein the agent is a fluorescent tag.

11) The process of claim 1 wherein the agent is a radioactive tag.

12) The process of claim 1 wherein the agent is a colorimetric tag.

13) The process of claim 1 wherein the agent is an enzyme tag.
